(19) United States
(12) Patent Application Publication
(10) Pub. No.: US 2022/0346852 A1
(43) Pub. Date: Nov. 3, 2022

(54) CRYOTHERAPY AND CRYOABLATION SYSTEMS AND METHODS FOR TREATMENT OF TISSUE

(71) Applicant: The General Hospital Corporation, Boston, MA (US)

(72) Inventors: Richard Rox Anderson, Boston, MA (US); Lilit Garibyan, Newton, MA (US); Emilia Javorsky, Watertown, MA (US); William Farinelli, Boston, MA (US)

(73) Assignee: The General Hospital Corporation, Boston, MA (US)

(21) Appl. No.: 17/861,138

(22) Filed: Jul. 8, 2022

Related U.S. Application Data
(63) Continuation of application No. 16/327,266, filed on Feb. 21, 2019.
(60) Provisional application No. 62/381,231, filed on Aug. 30, 2016.

(51) Int. Cl. A61B 18/02

(52) U.S. Cl. CPC A61B 18/0218; A61B 18/02; A61B 2018/00101

(57) ABSTRACT

Systems and methods for the use of cooling to trigger desirable effects of increased vasculature and/or development of new collagen in biological tissue are provided. In particular, the systems and methods provide a cooling treatment system configured to provide bulk or fractionated cooling at either at ablative temperatures or intermediary remodeling temperatures to promote tissue remodeling by inducing increased vasculature and/or the formation of new collagen.

### CROSS-REFERENCES TO RELATED APPLICATIONS

This application is a continuation of U.S. patent application Ser. No. 16/327,266, filed Feb. 21, 2019, which is a national stage filing under 35 U.S.C. § 371 of international application number PCT/US2017/048995, filed Aug. 29, 2017, which claims priority under 35 U.S.C. § 119(e) to U.S. Provisional Patent Application Ser. No. 62/381,231, filed Aug. 30, 2016, the entire contents of each of which are incorporated herein by reference.

### STATEMENT REGARDING FEDERALLY SPONSORED RESEARCH

Not Applicable.

### BACKGROUND

The disclosure relates generally the therapeutic use of cooling and, more specifically, to cryotherapy and cryoablation systems and methods for the treatment of tissue.

Controlled cooling and/or heating of biological tissue, such as skin tissue, can produce various therapeutic effects. For example, heating has been shown to improve skin defects by the application of electromagnetic radiation to induce thermal injury to the skin. The thermal injury results in a complex wound healing response of the skin, which can lead to biological repair of the injured skin, and may be accompanied by other desirable effects.

Skin tissue cooling has been implemented in hypopigmentation and tissue reshaping applications. Certain tissue cooling procedures and devices, such as conventional cryoprobes, can cause cryoinjury, or wound to the tissue, and generate cellular damage (i.e., cryoablation). Similar to the thermal injury, cryoinjury can trigger a complex wound healing process, which can lead to biological repair of the skin. Other tissue cooling techniques may implement temperatures that do not induce cryoinjury, but still stimulate a therapeutic effect as a result of exposure to the cold temperature (i.e., cryotherapy).

### BRIEF SUMMARY

The present disclosure provides systems and methods for the use of cooling to trigger desirable effects, such as increased vasculature and/or development of new collagen in biological tissue. In particular, the systems and methods provide a cooling treatment system configured to provide bulk or fractionated cooling at either ablative temperatures or intermediary remodeling temperatures to promote tissue remodeling by inducing increased vasculature and/or the formation of new collagen.

In one aspect, the present disclosure provides a method for causing angiogenesis in a subject. The method includes identifying treatment parameters for a desired tissue region of the subject for receiving a treatment including cooling, using a cooling device, to a desired temperature provided by the cooling device. The treatment parameters are based in part on at least one of the desired treatment tissue region or the treatment. The method further includes applying the treatment using the treatment parameters, and eliciting an angiogenesis response of the desired treatment tissue to the treatment.

In another aspect, the present disclosure provides a method for causing collagen remodeling in a subject. The method includes identifying treatment parameters for a desired tissue region of the subject for receiving a treatment including cooling, using a cooling device, to a desired temperature provided by the cooling device. The treatment parameters are based in part on at least one of the desired treatment tissue region or the treatment. The method further includes applying the treatment using the treatment parameters, and eliciting a collagen remodeling response of the desired treatment tissue to the treatment.

In yet another aspect, the present disclosure provides a method for causing cryolipolysis in a subject. The method includes identifying treatment parameters for a desired tissue region of the subject for receiving a treatment including cooling, using a cooling device, to a desired temperature provided by the cooling device. The treatment parameters are based in part on at least one of the desired treatment tissue region or the treatment, and the desired temperature is between approximately minus 200 degrees Celsius and approximately 30 degrees Celsius. The method further includes applying the treatment using the treatment parameters, and eliciting a cryolipolysis response of the desired treatment tissue to the treatment.

In still another aspect, the present invention provides a cooling treatment system for applying cooling therapy to a desired tissue region of a patient. The cooling treatment system includes a cooling device, and a delivery device configured to be cooled by the cooling device and subject the desired tissue region to a desired temperature provided by the cooling device. The desired temperature is between approximately minus 200 degrees Celsius and approximately 30 degrees Celsius.

The foregoing and other aspects and advantages of the invention will appear from the following description. In the description, reference is made to the accompanying drawings which form a part hereof, and in which there is shown by way of illustration a preferred embodiment of the invention. Such embodiment does not necessarily represent the full scope of the invention, however, and reference is made therefore to the claims and herein for interpreting the scope of the invention.

### BRIEF DESCRIPTION OF DRAWINGS

The invention will be better understood and features, aspects and advantages other than those set forth above will become apparent when consideration is given to the following detailed description thereof. Such detailed description makes reference to the following drawings.

FIG. 1 shows a cooling treatment system according to one aspect of the present disclosure.

FIG. 2 is a schematic illustration of the cooling treatment system of FIG. 1.

FIG. 3 shows the cooling treatment system of FIG. 1 including a warming unit, thermal imaging, and depth imaging according to another aspect of the present disclosure.

FIG. 4 is a schematic illustration of the cooling treatment system of FIG. 3.

FIG. 5 shows an interface and a delivery device of the cooling treatment system of FIG. 1 where the delivery device includes shorter protrusions according to one aspect of the present disclosure.

FIG. 6 shows an interface and a delivery device of the cooling treatment system of FIG. 1 where the delivery device includes longer protrusions according to one aspect of the present disclosure.

FIG. 7 shows an interface and a delivery device of the cooling treatment system of FIG. 1 where the delivery device defines a larger area and includes shorter protrusions according to one aspect of the present disclosure.

FIG. 8 shows an interface and a delivery device of the cooling treatment system of FIG. 1 where the delivery device defines a larger area and includes longer protrusions according to one aspect of the present disclosure.

FIG. 9 shows an interface and a delivery device of the cooling treatment system of FIG. 1 where the delivery device defines an arcuate shape according to one aspect of the present disclosure.

FIG. 10A shows a delivery device of the cooling treatment system of FIG. 1 where the delivery device defines a rod shape with protrusions extending from substantially half of a circumference of the rod according to one aspect of the present disclosure.

FIG. 10B is a top view of the delivery device of FIG. 10A.

FIG. 11A shows a delivery device of the cooling treatment system of FIG. 1 where the delivery device defines a rod shape with protrusions extending circumferentially around the rod according to one aspect of the present disclosure.

FIG. 11B is a top view of the delivery device of FIG. 11A.

FIG. 12 shows a protrusion of the cooling treatment system of FIG. 1 configured to be cooled by conduction according to one aspect of the present disclosure.

FIG. 13 shows a protrusion of the cooling treatment system of FIG. 1 having an insulating jacket according to one aspect of the present disclosure.

FIG. 14 shows a protrusion of the cooling treatment system of FIG. 1 configured to be actively cooling via a circulating cryogen according to one aspect of the present disclosure.

FIG. 15 shows a protrusion of the cooling treatment system of FIG. 1 where a proximal end of the protrusion is actively insulated/warmed according to one aspect of the disclosure.

FIG. 16A shows a plurality of protrusions of the cooling treatment system of FIG. 1 in the form of a plurality of needles configured to inject a slurry according to one aspect of the present disclosure.

FIG. 16B shows a plurality of protrusions of the cooling treatment system of FIG. 1 in the form of a plurality of needles coupled to a manifold and configured to inject a slurry according to one aspect of the present disclosure.

FIG. 17 shows a protrusion of the cooling treatment system of FIG. 1 in the form of a needle configured to inject a slurry in a bulk cooling pattern according to one aspect of the present disclosure.

FIG. 18 shows a protrusion of the cooling treatment system of FIG. 1 in the form of a needle having a cooling apparatus in a contracted state according to one aspect of the present disclosure.

FIG. 19 shows the protrusion of FIG. 18 with the cooling apparatus in an expanded state according to one aspect of the present disclosure.

FIG. 20 shows a protrusion of the cooling treatment system of FIG. 1 in the form of a needle having a plurality of tips configured to impart a fractional cooling pattern according to one aspect of the present invention.

FIG. 21 shows a protrusion of the cooling treatment system of FIG. 1 in the form of a needle having a plurality radially extending of tips configured to impart a fractional cooling pattern according to one aspect of the present invention.

FIG. 22 illustrates one non-limiting fractional cooling pattern achievable by the cooling treatment system of FIG. 1.

FIG. 23 illustrates one non-limiting example of an array bulk cooling pattern achievable by the cooling treatment system of FIG. 1.

FIG. 24 illustrates one non-limiting example of a bulk cooling pattern achievable by the cooling treatment system of FIG. 1 using a protrusion.

FIG. 25 illustrates one non-limiting example of a bulk cooling pattern achievable by the cooling treatment system of FIG. 1 following a fanning injection through a needle.

FIG. 26 is a flowchart outlining steps for operating a cooling treatment system to perform cryotherapy and/or cryoablation according to one aspect of the present disclosure.

FIG. 27 is a graph illustrating a thermal border as a function of post-slurry injection time for a subcutaneously injected cryoslurry in a rat.

FIG. 28 is a graph illustrating a skin temperature as a function of post-slurry injection time for a slurry with 10% ice content and a slurry with 50% ice content.

FIG. 29 is a graph illustrating a polynomial regression model used to fit temperature as a function of post-injection time for the first 60 second after the slurry injection.

FIG. 30 is a graph illustrating a quadratic regression model used to fit temperature as a function of post-injection time for slurry injection cooling and subsequent rewarming.

FIG. 31 is a graph illustrating skin surface temperature as a function of post-cooling time following an injection of a cooled needle array.

FIG. 32 is a contour plot of a temperature distribution on ex-vivo mouse skin using a fractional needle array cooled to −10° C.

FIG. 33 is a graph illustrating a temperature of ex-vivo mouse skin as a function of time at a location adjacent to a needle in a fractional needle array and surrounding tissue.

FIG. 34 illustrates an experimental setup for a single bulk slurry injection into human post abdominoplasty tissue.

FIG. 35 illustrates an experimental setup for a fractional slurry injection into human post abdominoplasty tissue.

FIG. 36 is a graph illustrating temperature in human post abdominoplasty tissue as a function of time measured at two locations laterally from an injection site for a single bulk slurry injection.

FIG. 37 is a graph illustrating temperature in human post abdominoplasty tissue as a function of time measured at two locations laterally from an injection site for a fractional slurry injection.

### DETAILED DESCRIPTION

Recent evidence suggests that the wound healing process triggered by damage to biological tissue (e.g., human skin) is distinctly different between heat injury and cryoinjury. For example, skin lesions tend to heal well with minimal to no scaring after controlled cryoinjury. Both thermal burns and freezing produce similar tissue destruction, but the resistance of collagen, fibroblasts, and connective tissue matrix to freezing are the basis of favorable healing. Though the tissues are devitalized by freezing, the matrix is usually little changed and the preservation of this architecture is important to repair.

Wound healing is an active process that begins with an inflammatory reaction at the border of the lesion. There is always a very brisk inflammatory response observed after freeze injury, which is postulated to help initiate the proper healing process and prevent any infection associated with the injury. The inflammatory cell infiltrate also contributes to the development of apoptosis and to tissue destruction. As granulation tissue forms, fibroblasts differentiate to myofribroblasts and damaged collagen is replaced by new collagen. The cellular infiltration helps establish new vasculature, which plays a critical role in the rapport of the devitalized tissue.

The systems and methods described herein leverage use of cooling to trigger the desirable effects of increased vasculature and/or the development of new collagen in biological tissue. In particular, the systems and methods provide a cooling treatment system configured to provide bulk or fractionated cooling in a precisely controlled manner at either at very cold ablative temperatures or intermediary remodeling temperatures to promote tissue remodeling by inducing increased vasculature and the formation of new collagen. Such a cooling treatment system can provide a device-based approach for treatment of a wide variety of unmet clinical needs that arise from decreased vasculature and/or decreased collagen. Additionally, the cooling treatment system can provide a safe, non-pharmacological treatment approach, and the tissue remodeling provided by the system can have long lasting effect. Further, the use of cooling can provide a cost-effective solution that can be provided to a wide range of medical facilities and by practitioners who may have been priced out of current energy based (e.g., laser) therapies.

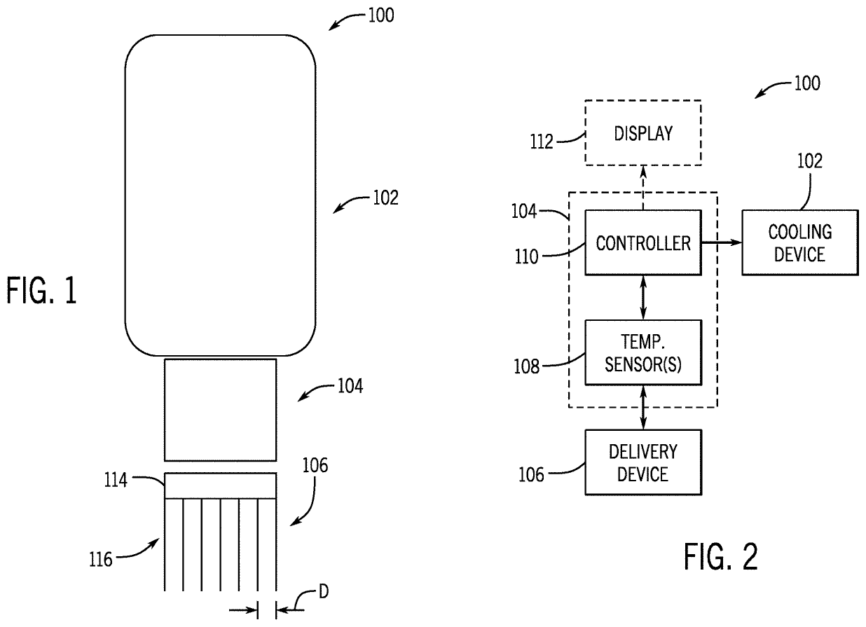

FIGS. 1 and 2 illustrate a cooling treatment system 100 according to one non-limiting example of the present disclosure. The cooling treatment system 100 includes a cooling device 102, an interface 104, and a delivery device 106. The cooling device 102 is configured to provide cooling through the interface 104 and to the delivery device 106. In some non-limiting examples, the cooling device 102 may be in the form of a thermoelectric cooler, cryogen gas, liquid nitrogen, liquid argon, cooled liquids, a Joule-Thomson refrigerator, nitrous oxide, and carbon dioxide, to name a few.

The interface 104 may be fabricated from a material with a high thermal conductivity to facilitate efficient heat transfer between the cooling device 102 and the delivery device 106. The interface 104 may be coupled to the cooling device 102 (e.g., via an adhesive or a mechanical coupling mechanism) and may be detachably coupled to the delivery device 106. The interface 104 may include one or more temperature sensors 108 and a controller 110. The temperature sensors 108 are configured to measure a temperature at one or more locations on the delivery device 106 and communicate the measured temperatures to the controller 110. The controller 110 is in communication with the cooling device 102 and may be configured to control a temperature output by the cooling device 102, thereby controlling a temperature of the delivery device 106. In one non-limiting example, a desired temperature of the delivery device 106 may be input to the controller 110 and the controller 110 may be configured to control the cooling device 102 to achieve the desired temperature of the delivery device 106, as measured by the temperature sensors 108. In some non-limiting examples, the controller 110 may in communication with a display 112 and configured to instruct the display 112 to display, for example, a temperature of the delivery device 106, a time to administer the delivery device 106, a depth of the delivery device 106, and/or a temperature of the surface of a desired tissue region.

The delivery device 106 includes a base 114 and a plurality of protrusions 116 extending from the base 114. In some non-limiting examples, the plurality of protrusions 116 may be in the form of a needle array configured to penetrate to a desired depth within a tissue region of a patient. As will be described below, in these non-limiting examples, the needle array may be configured to enable the injection of a slurry (i.e., a liquid and ice crystal mixture). In other non-limiting examples, the plurality of protrusions 116 may be in the form of a plurality of conductive posts, or pins, configured to engage a surface of a tissue region of a patient to provide topical cooling. It should be appreciated that although the illustrated delivery device 106 includes a plurality of protrusions 116, in other non-limiting example, the delivery device 106 may include one or more protrusions 116.

A distance D defined between adjacent pairs of the plurality of protrusions 116 may be dimensioned to ensure that a fractional cooling pattern may be achieved in or on a desired tissue region. That is, the distance D can be dimensioned such that discrete zones of cooling are achieved when the delivery device 106 is administered. In combination with the spacing of the plurality of protrusions 116, a time that the delivery device 106 is engaged with the desired tissue region can also define the resulting cooling pattern, as will be described below.

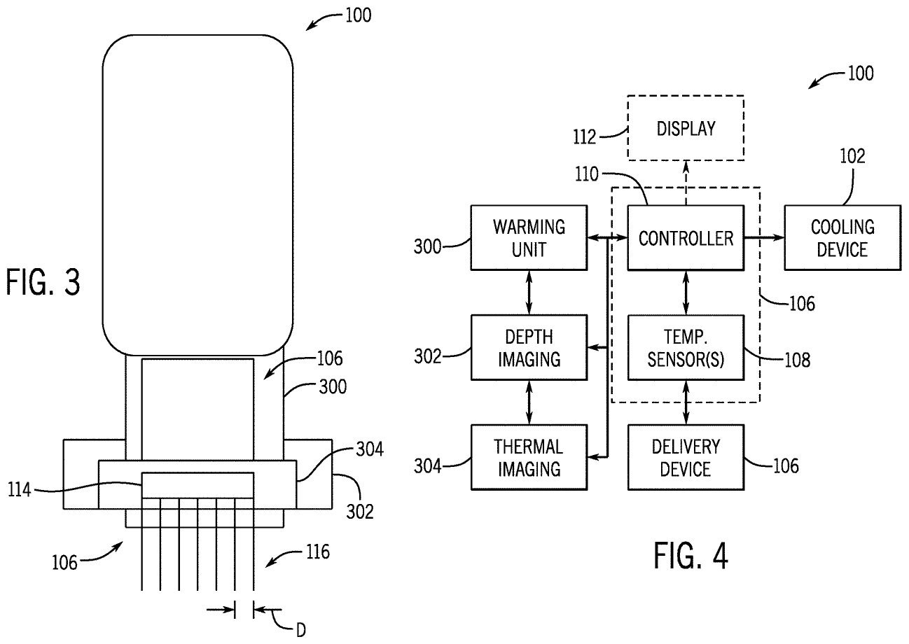

FIGS. 3 and 4 illustrate another non-limiting example of the cooling treatment system 100 according to the present disclosure. As shown in FIGS. 3 and 4, the cooling treatment system 100 may include a warming unit 300, a depth imaging device 302, and a thermal imaging device 304 each in communication with the controller 110. The warming unit 300 may be configured to provide selectively controlled warming, for example, to a proximal end of the plurality of protrusions 116. Selectively warming the proximal end of the plurality of protrusions 116 can enable only a distal end, or tip, of the plurality of protrusions 116 to provide cooling to a desired tissue area. Alternatively or additionally, the warming unit 300 may be configured to provide selective warming to a tissue surface (e.g., epidermis), and/or configured to provide selective warming to deeper tissue below a tissue surface (e.g., subcutaneous fat) via radio-frequency (RF) heating or laser heat.

The depth imaging device 302 may be configured to measure and image a depth that the plurality of protrusions 116 penetrate into a desired tissue region. The depth imaging device 302 may be configured to provide a measured depth of the plurality of protrusions 116 to the controller 110. Alternatively or additionally, the controller 110 may relay an image to the display 112 of the plurality of protrusions 116 penetrating into a desired tissue region to provide active feedback to a user of the cooling treatment system 100. In some non-limiting examples, the depth imaging device 302 may be in the form of an OCT imaging device, magnetic resonance imaging (MRI) device, an ultrasound device, or an X-ray device.

The thermal imaging device 304 may be configured to measure and image a temperature at a surface of a desired tissue region. That is, when the plurality of protrusions 116 are applying cooling on or in a desired tissue region, the thermal imaging device 304 may enable a user to visually inspect a temperature at a surface of the desired tissue region. This can enable a user to ensure a desired cooling pattering is achieved (i.e., fractionated vs. bulk cooling) and/or verify a desired temperature is applied (i.e., ablative vs. cryostimulatory/cryotherapy) to the desired tissue region. In some non-limiting examples, the thermal imaging device 304 may be integrated into the cooling treatment system 100 and may be in communication with the controller 110. The controller may relay a thermal image acquired by the thermal imaging device 304 to the display 112 to provide active feedback to a user of the cooling treatment system 100. In some non-limiting examples, the thermal imaging device 304 may be a separate component used or worn by a user of the cooling treatment system 100 while providing cooling on or in a desired tissue region. In some non-limiting examples, the thermal imaging device 304 may be in the form of an infrared camera, thermal imaging glasses, or a mobile device with a thermal imaging add-on. In other non-limiting examples, the thermal imaging device 304 may comprise one or more thermocouples (or other thermal sensors), or infrared temperature sensing device.

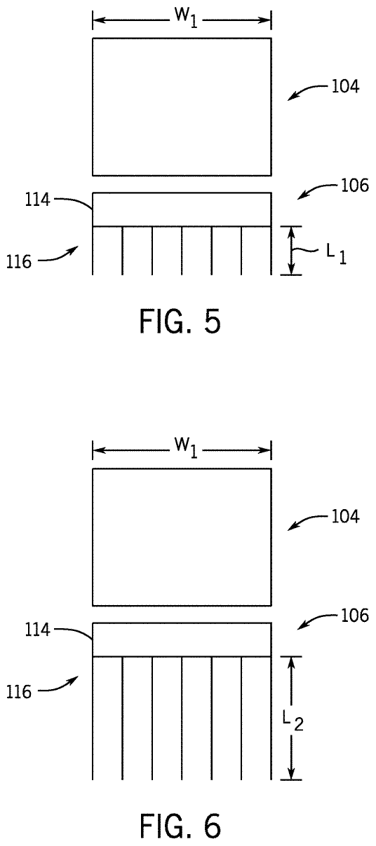

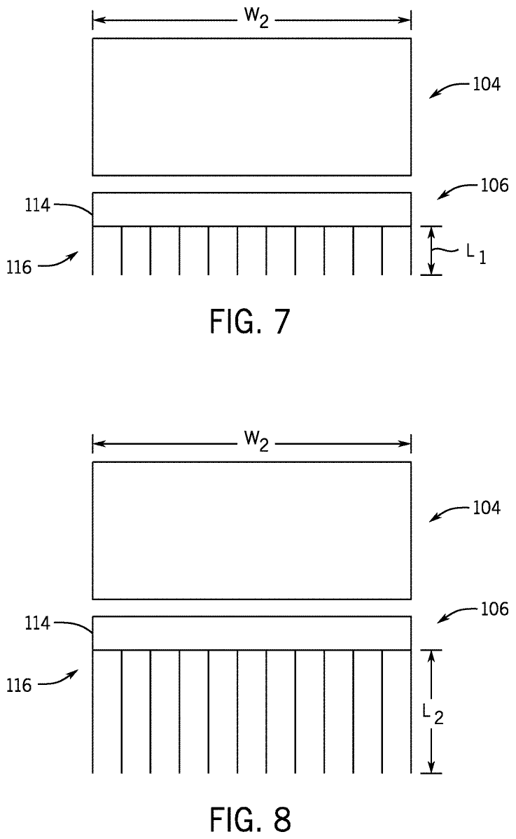

It should be appreciated that the delivery device 106 and the plurality of protrusions 116 arranged thereon may define alternative shapes and sizes for a given tissue application. For example, as shown in FIGS. 5-8, the delivery device 106 and the corresponding interface 104 may define different treatment areas and/or different depths of treatment. In some non-limiting examples, the base 114 of the delivery device 106 and the corresponding interface 104 may define a width W₁. In other non-limiting examples, the base 114 of the delivery device 106 and the corresponding interface 104 may define a width W₂, where W₂ is greater than W₁. In some non-limiting examples, the plurality of protrusions 116 may each define a length L₁. In other non-limiting examples, the plurality of protrusions 116 may each define a length L₂, where L₂ is greater than L₁. It should also be appreciated that a density (i.e., the number of the plurality of protrusions 116 extending from the delivery device 106) may be varied, for example, by altering the distance D between adjacent pairs of the plurality of protrusions 116 and accordingly adding or subtracting protrusions to the delivery device 106. These alternative geometric configurations may be tailored to provide desired treatment parameters for a given application of the cooling treatment system 100.

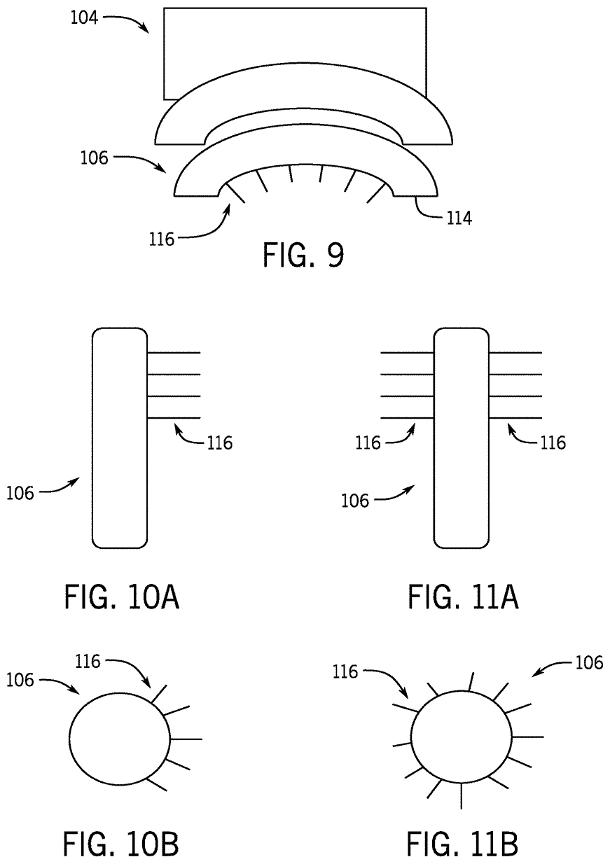

The illustrated base 114 of the delivery device 106 of FIGS. 1, 3, and 5-8 defines a generally flat profile, which results in the plurality of protrusions defining a generally flat treatment profile. In other non-limiting examples, as shown in FIGS. 9-11, the delivery device 106 may define alternative shapes and profiles to accommodate various anatomical locations on a patient. As shown in FIG. 9, in some non-limiting examples, the base 114 of the delivery device 106 may define a generally arcuate shape, which thereby arranges the plurality of protrusions 116 in a corresponding arcuate treatment profile.

Turning to FIGS. 10A-11B, in some non-limiting examples, the delivery device 106 may be in the form of a wand, or rod, shape with the plurality of protrusions 116 extending from a distal end thereof. As shown in FIGS. 10A and 10B, in one non-limiting example, the plurality of protrusions 116 may extend radially outward from the distal end of the delivery device 106. The plurality of protrusions 116 may be arranged partially circumferentially around a periphery of the delivery device 106. That is, the plurality of protrusions 116 may be arranged circumferentially around approximately half (e.g., between 0 degrees and 180 degrees) of the delivery device 106. As shown in FIGS. 11A and 11B, in one non-limiting example, the plurality of protrusions 116 may extend radially from the distal end of the delivery device 106, and may be arranged circumferentially around an entirety of the periphery of the delivery device 106 in approximately equal increments. Alternatively or additionally, the plurality of protrusions 116 may be arranged circumferentially around the periphery of the delivery device 106 in non-equal increments. In the non-limiting examples of FIGS. 10A-11B, the plurality of protrusions 116 may be retractably received within the delivery device 106. For example, the delivery device 106 may be inserted into the target tissue with the plurality of protrusions 106 retracted into the delivery device 106 and then the plurality of protrusions 106 may be deployed from the delivery device 106 one within the target tissue.

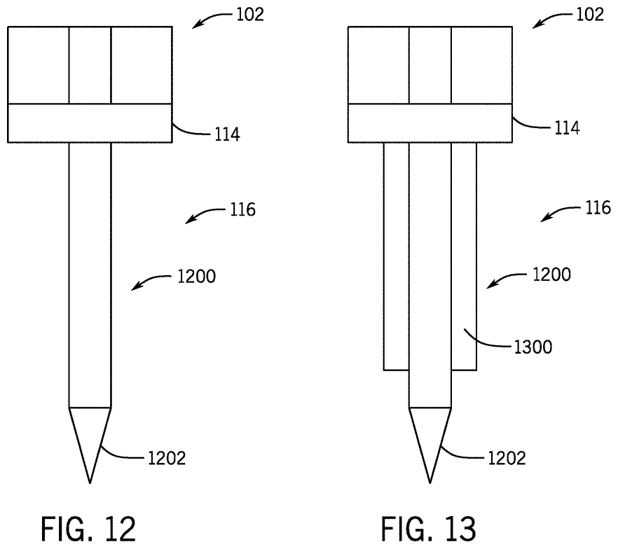

FIG. 12 illustrates one non-limiting example of one of the plurality of the plurality of protrusions 116 according to one aspect of the present disclosure. The illustrated protrusion 116 is in the form of a needle 1200 including a needle tip 1202 arranged at a distal end thereof. The needle 1200 can be fabricated from a metal material and the entire axial length of the needle 1200 can be cooled via conduction from the cooling device 102. The needle 1200 may be sized to be between approximately 15 gauge and approximately 35 gauge or smaller. In some non-limiting examples, as shown in FIG. 13, the needle 1200 may include insulation 1300 wrapped around a desired axial length of the needle 1200. That is, the insulation 1300 may extend axially along the needle 1200 while leaving the needle tip 1202 of the needle 1200 uninsulated. This, along with an axial length defined by the needle 1200, can control a depth within a desired tissue region that the cooling is applied. Further, only providing cooling at the needle tip 1202 can prevent healthy tissue from being damaged by the cooling applied at the needle tip 1202. In other non-limiting examples, the insulation 1300 may be replaced by an active warming unit wrapped around the needle 1200. Similar to the insulation 1300, the active warming unit may not be arranged around the needle tip 1202 enabling the cooling to be applied to a desired tissue region at a target depth defined by the axial length of the needle 1200.

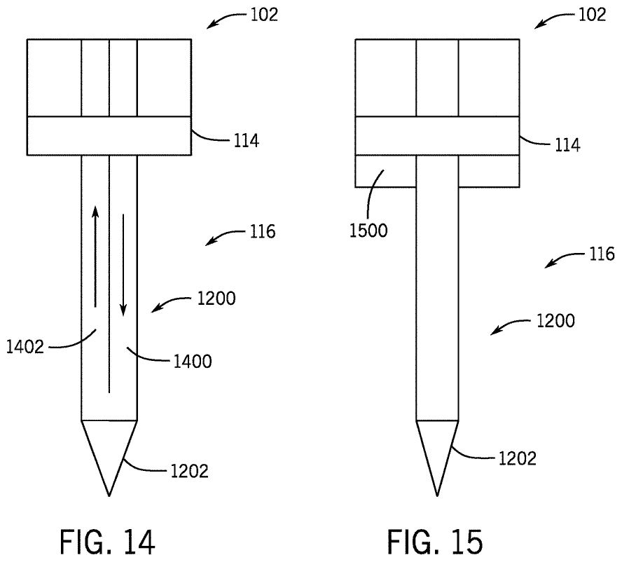

In some non-limiting examples, as shown in FIG. 14, the entire axially length of the needle 1200 may be actively cooled by a circulated cryogen. The illustrated needle 1200 may include an inlet passage 1400 and an outlet passage 1402 arranged within the needle 1200 and extending axially along the needle 1200. A cryogen may be circulated into the inlet passage 1400 and out of the outlet passage 1402 to actively cool the entire axial length of the needle 1200.

In some non-limiting examples, as shown in FIG. 15, a warming unit 1500 may be arranged adjacent to a proximal end of the needle 1200. The warming unit 1500 may be configured to apply warming to a surface (e.g., epidermis) of a desired tissue region. This can prevent healthy tissue from being damaged by the cooling applied by the needle 1200.

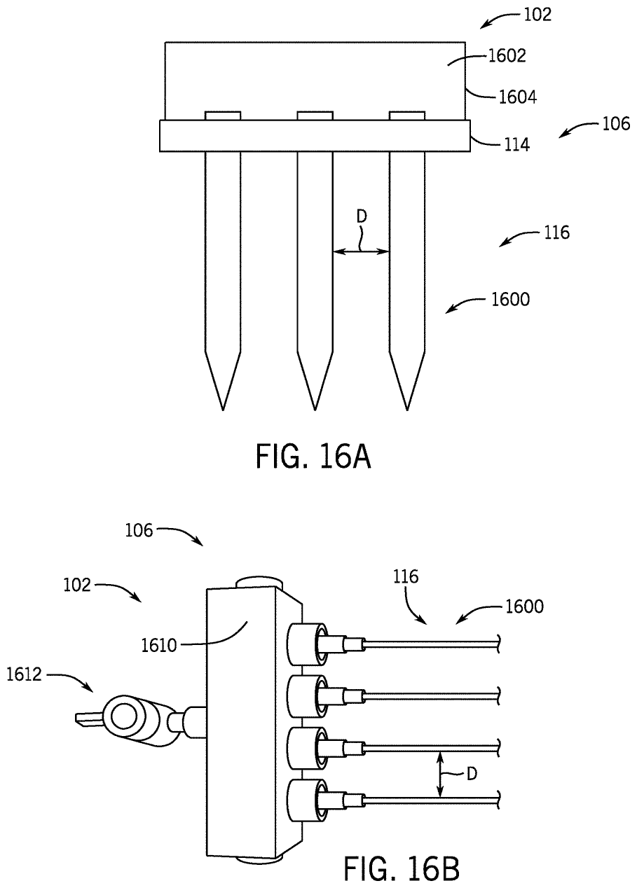

As described above, in some non-limiting examples, the plurality of protrusions 116 may be configured to inject a desired volume of slurry into a desired tissue region to apply cryotherapy or cryoablation. FIG. 16A illustrates one non-limiting example of the plurality of protrusions 116 in the form of a needle array 1600 configured to inject a slurry 1602 into a desired tissue region. The needles of the needle array 1600 may be sized to be between approximately 15 gauge and approximately 30 gauge. The slurry 1602 can be arranged in a cartridge 1604, which can be removably coupled to the delivery device 106. The slurry 1602 may be prepared to achieve a desired cooling temperature and to contain appropriately sized ice crystals to ensure fluid flow through the needle array 1600, as will be described below. Additionally, a volume of slurry injected and/or the distance D between adjacent pairs of needles 1600 may be designed to ensure a desired cooling pattern is achieved (i.e., fractionated vs. bulk cooling).

In another non-limiting example, as illustrated in FIG. 16B, the needle array 1600 may be removably coupled to a manifold 1610. Each of the needles in the needle array 1600 may be removably coupled to the manifold 1610, for example, by a threaded engagement, a quick disconnect fitting, or a push-on fitting. The removable coupling of the needle array 1600 to the manifold 1610 enables the number and/or arrangement of the needles in the needle array 1600 to be modified by the user, as desired. Alternatively or additionally, the same manifold 1610 may be used to perform injections with needles of varying sizes (e.g., a 15 gauge needle array vs. a 30 gauge needle array). Alternatively or additionally, a spacing between adjacent needles in the needle array 1600 may be controlled by the number and/or orientation of the needles that are coupled to the manifold 1610.

In the illustrated non-limiting example, the manifold 1610 is coupled a needle array 1600 comprising four needles. In other non-limiting examples, the manifold 1610 may be coupled to a needle array 1600 comprising more or less than four needles arranged in any pattern as desired.

The manifold 1610 includes an inlet port 1612 that is configured to be removably coupled to a slurry injection device (not shown). The manifold 1610 may include internal passageways that provide fluid communication between the inlet port 1612 and each of the needles in the needle array 1600. The slurry injection device may, for example, be in the form of a syringe-type device that includes a desired volume of slurry to be injected into a desired tissue region. In some non-limiting examples, the syringe-type device may be manually actuatable to facilitate the injection of the slurry. In some non-limiting examples, the syringe-type device may be electronically controlled (e.g., like a syringe pump) to facilitate the injection of the slurry at a predetermined fluid flow rate.

In operation, for example, a user may install the desired size and arrangement of needle array onto the manifold 1610 and, subsequently, couple the slurry injection device, which is filled with a desired volume of slurry, to the inlet port 1612. With the delivery device 102 assembled, a user may inject the needle array 1600 into a desired tissue region to a desired depth within the desired tissue region, and inject the slurry to achieve a fractional cooling pattern within the desired tissue region.

In some non-limiting applications, the fractional slurry injection capabilities of the delivery device 102 of FIGS. 16A and 16B may be able to cover a larger area of target tissue when compared with a single injection of an equivalent slurry volume. For example, a fractional slurry injection device may be able to cover approximately double the area of target tissue with a single slurry injection, when compared to bulk cooling with a single injection. The fractional slurry injection capabilities of the delivery device 102 may provide several other operational and functional advantages, when compared to a single bulk injection of an equivalent slurry volume. For example, reduced injection force required to deliver the slurry into the target tissue, reduced time required to deliver the slurry into the target tissue (e.g., approximately half of the time when compared to a single injection), more uniform spread of slurry into the target tissue, and reduced probability of affecting blood vessels and pain. In some non-limiting applications, the more uniform spread of slurry into the target tissue may translate to a more uniform reduction of fat within the target tissue thereby avoiding an unwanted side effect of forming dents or depressions in the target tissue. In some cases, a single injection within a large amount of slurry may create a bulge/swelling and tension within the target tissue, which can lead to ruptured blood vessels and bruising. A large single injection may also stretch subcutaneous nerves and cause pain. These undesirable characteristics of a single injection may be avoided by the use of a fractional slurry injection, which can deliver, for example via the delivery device 102, a more uniform distribution of slurry in smaller aliquots into the target tissue.

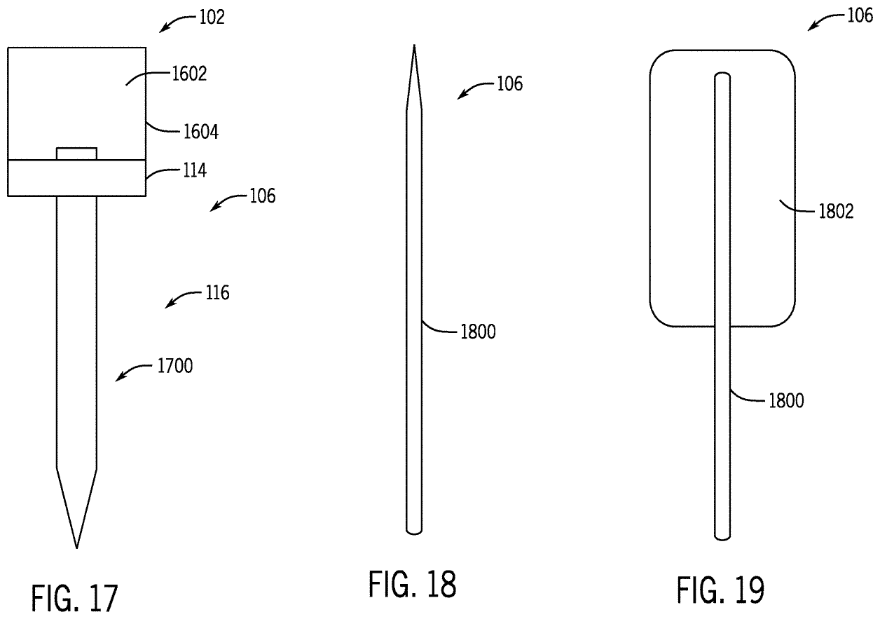

In one non-limiting example, as opposed to the needle array 1600, the cooling treatment system 100 may implement a single needle 1700, as shown in FIG. 17. The needle 1700 may be sized to be between approximately 15 gauge and approximately 35 gauge or smaller. In this non-limiting example, the cooling treatment system 100 may be configured to provide bulk cooling to a desired tissue region.

In some non-limiting examples, as shown in FIGS. 18 and 19, the delivery device 106 may comprise an expandable needle 1800 as opposed, or in addition with, to the plurality of protrusions 116. The expandable needle 1800 may be cooled by the cooling device 102 and subsequently be advanced by a user of the cooling treatment system 100 to a desired tissue region (e.g., lipid rich tissues in a patient's tongue/airway). Once the expandable needle 1800 reaches the desired tissue region, the user can expand a balloon 1802 attached to the expandable needle 1800. A slurry at a desired temperature may then be delivered through the expandable needle 1800 to the balloon 1802 to provide cooling to the desired tissue region. It should be appreciated that the balloon 1802 may not need to be inflated prior to injection of the slurry. Rather, injection of the slurry may inflate the balloon 1802. Once the desired cooling treatment has been applied to the desired tissue region, the balloon 1802 may be retracted to in deflated state (FIG. 18).

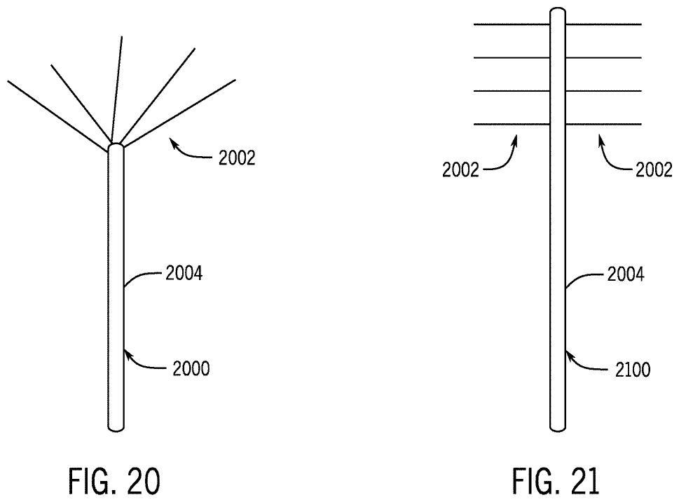

FIGS. 20 and 21 illustrate two non-limiting examples of fractional delivery arrays 2000 and 2100, which may be implement in the delivery device 106 as opposed to, or in addition with, the plurality of protrusions 116. The fractional delivery array 2000 may be advanced by a user of the cooling treatment system 100 to a desired tissue region (e.g., lipid rich tissues in a patient's tongue/airway). Once the fractional delivery array 2000 is advanced to the desired tissue region, a slurry may be delivered to the desired tissue region in a fractional pattern through a plurality of needles 2002. The plurality of needles 2002 can extend outwardly from a distal end of an array tube 2004. As shown in FIGS. 20 and 21, the plurality of needles 2002 may be arranged in alternative patterns to define alternative fractional cooling patterns, as desired.

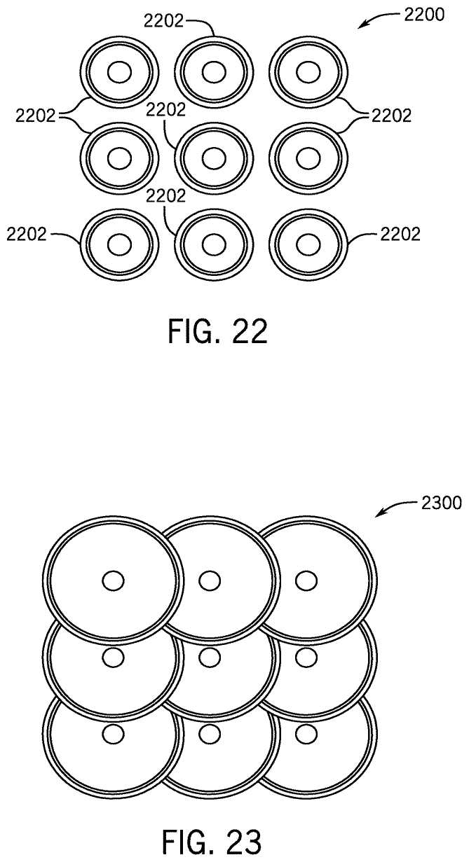

As described above, the cooling treatment system 100 may be designed to provide a desired cooling pattern. That is, in one non-limiting example, the cooling treatment system 100 may be designed to provide a fractional cooling pattern to a desired tissue region. FIG. 22 illustrates one non-limiting example of a fractional cooling pattern 2200, which may be achieved via the injection of a slurry, topical cooling, or the injection of actively cooled needles, as described above with reference to the delivery device 106. As shown in FIG. 22, discrete cooling zones 2202 are present with area of untreated tissue arranged between adjacent cooling zones in the fractional pattern 2200. It should be appreciated that the number of discrete cooling zones 2202 shown in FIG. 22 is meant for purposes of illustration and is not meant to be limiting in any way. In some non-limiting examples, the cooling treatment system 100 may be configured to provide ablative cooling therapy (i.e., cryoablation) in a fractional pattern at a temperature between approximately −180° C. and approximately −20° C. In some non-limiting examples, the cooling treatment system 100 may be configured to provide non-ablative cooling therapy (i.e., cryotherapy) in a fraction pattern at a temperature between approximately −20° C. and 5° C.

FIG. 23 illustrates an array bulk cooling pattern 2300 achievable by the cooling treatment system 100 according to one non-limiting example of the present disclosure. The illustrated array bulk cooling pattern 2300 can be formed via application of a cooling array (e.g., the plurality of protrusions 116, the needle array 1600, the plurality of needles 2002, etc.), which may be achieved via the injection of a slurry, topical cooling, or the injection of actively cooled needles, as described above with reference to the delivery device 106. As shown in FIG. 23, the array bulk cooling pattern 2300 defines a substantially uniform cooling profile over the desired tissue region. In some non-limiting examples, the cooling treatment system 100 may be configured to provide non-ablative cooling therapy (i.e., cryotherapy) in an array bulk cooling pattern at a temperature between approximately −20° C. and 5° C.

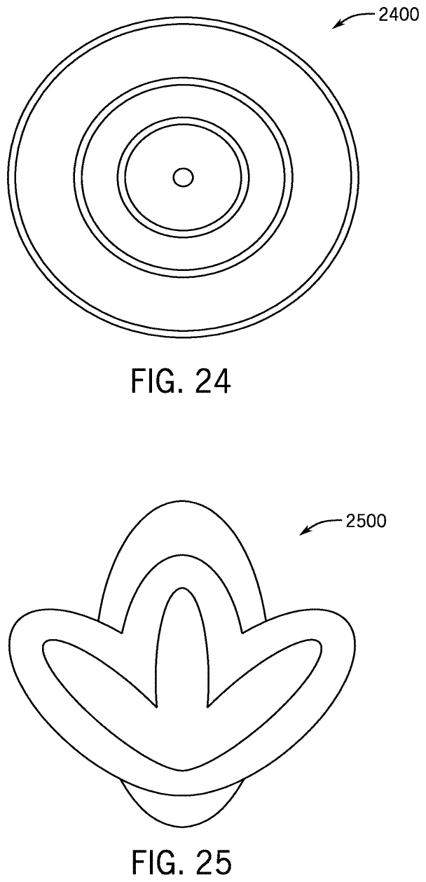

FIG. 24 illustrates a depot bulk cooling pattern 2400 achievable by the cooling treatment system 100 according to one non-limiting example of the present disclosure. The illustrated depot bulk cooling pattern 2400 may by formed via injection of a slurry from a single injection (e.g., the single needle 1700). The depot bulk cooling pattern 2400 defines concentric zones of cooling decreasing in temperature as they extend radially outwards from a center of the depot bulk cooling pattern 2400. It should be appreciated that alternative bulk cooling patterns are achievable by the cooling treatment system 100. For example, as shown in FIG. 25, the single needle 1700 may be configured to provide a fanning bulk cooling pattern 2500 when injection a slurry into a desired tissue region.

Operation and application of the cooling treatment system 100 will be described with reference to FIGS. 1-26. In application, the cooling treatment system is configured to provide bulk or fractionated cooling at either at very cold ablative temperatures or intermediary remodeling temperatures to promote tissue remodeling by inducing increased vasculature (i.e., angiogenesis) and the formation of new collagen (i.e., collagen remodeling). As will be described, there are various medical instances where a lack of blood flow and/or collagen formation can lead to a certain malady. Thus, the cooling treatment system 100 can be implemented to induce the formation of collagen and angiogenesis and thereby promote healing or treatment of the specific malady. In some non-limiting examples, the cooling treatment system 100 may be configured to subject a desired tissue region of a subject to a temperature between approximately −200° C. and approximately 30° C. In some non-limiting examples, the cooling treatment system 100 may be configured to subject a desired tissue region of a subject to a temperature between approximately −180° C. and approximately 30° C. In some non-limiting examples, the cooling treatment system 100 may be configured to subject a desired tissue region of a subject to a temperature between approximately −160° C. and approximately 30° C. In some non-limiting examples, the cooling treatment system 100 may be configured to subject a desired tissue region of a subject to a temperature between approximately −140° C. and approximately 30° C. In some non-limiting examples, the cooling treatment system 100 may be configured to subject a desired tissue region of a subject to a temperature between approximately −120° C. and approximately 30° C. In some non-limiting examples, the cooling treatment system 100 may be configured to subject a desired tissue region of a subject to a temperature between approximately −100° C. and approximately 30° C. In some non-limiting examples, the cooling treatment system 100 may be configured to subject a desired tissue region of a subject to a temperature between approximately −80° C. and approximately 30° C. In some non-limiting examples, the cooling treatment system 100 may be configured to subject a desired tissue region of a subject to a temperature between approximately −70° C. and approximately 30° C. In some non-limiting examples, the cooling treatment system 100 may be configured to subject a desired tissue region of a subject to a temperature between approximately −60° C. and approximately 30° C. In some non-limiting examples, the cooling treatment system 100 may be configured to subject a desired tissue region of a subject to a temperature between approximately −50° C. and approximately 30° C. In some non-limiting examples, the cooling treatment system 100 may be configured to subject a desired tissue region of a subject to a temperature between approximately −40° C. and approximately 30° C. In some non-limiting examples, the cooling treatment system 100 may be configured to subject a desired tissue region of a subject to a temperature between approximately −30° C. and approximately 30° C. In some non-limiting examples, the cooling treatment system 100 may be configured to subject a desired tissue region of a subject to a temperature between approximately −20° C. and approximately 30° C. In some non-limiting examples, the cooling treatment system 100 may be configured to subject a desired tissue region of a subject to a temperature between approximately −20° C. and approximately 20° C. In some non-limiting examples, the cooling treatment system 100 may be configured to subject a desired tissue region of a subject to a temperature between approximately −20° C. and approximately 10° C. In some non-limiting examples, the cooling treatment system 100 may be configured to subject a desired tissue region of a subject to a temperature between approximately −20° C. and approximately 5° C.

In one non-limiting example, bulk cooling may be applied by the cooling treatment system 100 for the purpose of inducing angiogenesis and collagen remodeling. This can be achieved via topical cooling (e.g., with the plurality of protrusions 116), slurry injection (e.g., with the plurality of protrusions 116, the needle array 1600, or the single needle 1700), or cryoneedles (e.g., with the plurality of protrusions 116). Alternatively, fractional cooling may be applied by the cooling treatment system 100 for the purpose of inducing angiogenesis and/or collagen remodeling. The induced collagen remodeling and angiogenesis provided by the application of the cooling treatment system 100 may be applied to any ischemic organ or tissue and/or a tissue experiencing laxity. The application of the cooling treatment system 100 to these tissues/organs may be used for the treatment of various ischemic diseases, such as, diabetic peripheral neuropathy, male pattern baldness, wound healing, skin aging, vaginal rejuvenation, onychomycosis, scar remodeling, revascularization of ischemic tissue/organ (i.e., nerve, muscle, skin, liver, kidney, heart, etc), treatment of lipomas and cellulite etc. Alternatively it should be appreciated that, in some application, the treatment provided by the cooling treatment system 100 may be combined with traditional pharmacologic agents that increase bloody supply or improve collagen remodeling.

In some applications, the cooling treatment system 100 may be used to selectively target lipid rich tissues in a patient's tongue or airways to induce cryolipolysis (the destruction of fat due to selective cold injury). This use of the cooling treatment system 100 may be used to treat obstructive sleep apnea (OSA), as the excess fat in the tongue/airway of the patient may be reduced via the selective application of cooling (e.g., via the application of the expandable needle 1800, or either one of the fractional delivery arrays 2000 and 2100). Alternatively or additionally, the selective application of cooling to the tongue/airway may initiate collagen remodeling in the airway that may improve the airway laxity associated with OSA.

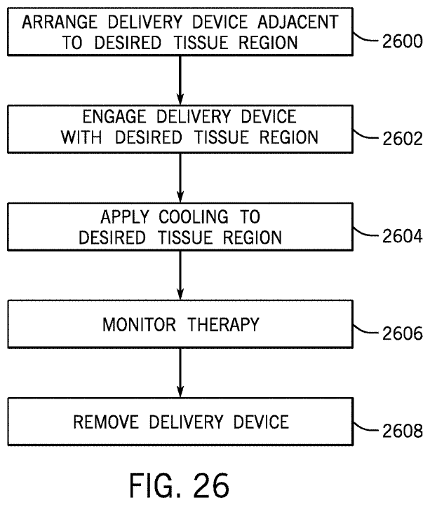

FIG. 26 illustrates one non-limiting example of steps for operating the cooling treatment system 100. As shown in FIG. 26, initially, at step 2600, a delivery device can be arranged adjacent to a desired tissue region where cooling treatment is desired to be applied. The delivery device may be any of the delivery devices described above, for example, the plurality of protrusions 116 (in the form of the needle 1200, the needle array 1600, or the single needle 1700, etc.), the expandable needle 1800, or the fractional delivery arrays 2000 or 2100. Once the delivery device is placed at step 2600, the delivery device can be brought into engagement with the desired tissue region at step 2602. The engagement of step 2602 can be a topical engagement, or an injection of a needle using any of the various delivery devices, described above. If a needle is injected at step 2602, the depth of injection can be controlled, for example, via the monitoring of the needle using the depth imaging device 302, by insulating the injected needle except for a needle tip, or actively warming tissue, as described above.

Once the delivery device is brought into engagement with the desired tissue region at step 2602, the cooling treatment system 100 can apply cooling to the desired tissue region at step 2604. The cooling applied at step 2604 may be either at cryoablative temperature or non-ablative, cryostimulatory temperatures, as described above. Additionally, the cooling applied at step 2604 may be topically applied via conductive cooling, via the injection of one or more conductively cooled needles, or via the injection of a cryoslurry from one or more needles utilizing any of the delivery devices described above. Further, the cooling applied at step 2604 may be in a bulk cooling pattern or a fractionated cooling pattern, as desired.

While the cooling is being applied at step 2604, a user (typically a trained medical professional) may monitor cooling therapy being applied at step 2606. The user may monitor the cooling therapy, for example, using the thermal imaging device 304, described above. The user may monitor the cooling therapy to ensure that the desired cooling pattern is being achieved. Alternatively or additionally, the user may monitor the cooling therapy to ensure that a desired temperature is being applied to the desired tissue region and/or to ensure that surrounding healthy tissue is not be subjected to potentially damaging temperatures.

The user can monitor the cooling therapy 2606 until they determine the desired therapeutic effect has been induced. Subsequently, the user can remove the delivery device at step 2608. It should be appreciated that the cooling therapy may be applied in numerous cycles at a specific time interval between cycles. In these instances, the steps from 2602-2608 may be repeated one or more times until the desired therapeutic effect has been induced.

### EXAMPLES

The following examples set forth, in detail, ways in which the cooling treatment system 100 may be used or implemented, and will enable one of skill in the art to more readily understand the principles thereof. The following examples are presented by way of illustration and are not meant to be limiting in any way.

The following data pertains to rat experiments performed in vivo. All temperature measurements were obtained using FLIR ONE non-contact thermal imaging.

Injection of CryoSlurry Subcutaneously

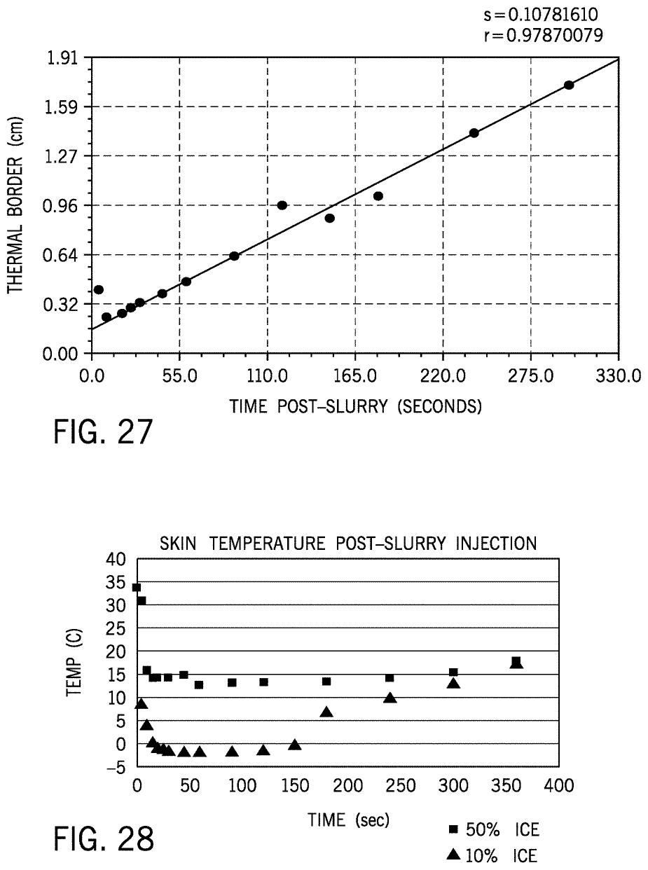

A slurry composition of normal saline mixed with 10% (by volume) Glycerol was prepared and injected subcutaneously into rats. The temperature range of the prepared slurry was between −3.5° C. and −2.5° C., and in the injection volume was 10 milliliters (mL). A thermal border created by the slurry injection was measured as a function of time post-injection. FIG. 27 illustrates the size of the thermal border as a function if post-injection time. As shown in FIG. 27, the size of the thermal border varies generally linearly with post-injection time. From the data in FIG. 27, an area of cooling can be approximated. This correlation, coupled with the fact that temperatures above approximately 14° C. (crystallization point of lipids) signify the end of a therapeutic effect of the cooling, can be used to approximate a cooling area and a minimum inter-injection distance to maintain a fractionated cooling pattern.

Table 1 below illustrates approximated data based on the experimental results for a 10 mL injection of −2.8° C. slurry with 50% ice content (by volume).

TABLE 1
Injection Estimates Based on 10 mL of −2.8°
C. slurry with 50% ice content

| | | Minimum Inter- | |
|---|---|---|---|
| | Radius | Injection Distance to | |
| Time Post | of Thermal | maintain fractionated | Cooling |
| Cooling (sec) | Border (cm) | pattern (cm) | Area (cm2) |
| 10 | 0.21 | 0.42 | 0.14 |
| 30 | 0.32 | 0.64 | 0.32 |
| 60 | 0.48 | 0.96 | 0.72 |
| 120 | 0.80 | 1.60 | 1.99 |
| 180 | 1.11 | 2.22 | 3.90 |
| 240 | 1.43 | 2.86 | 6.44 |
| 300 | 1.75 | 3.00 | 9.62 |

Estimated Skin Temperature Post Slurry Injection

A skin temperature post-slurry injection was estimated using the data from Table 1, above, for a 10 mL slurry injection at −2.8° C. with 50% ice content (by volume). As described above, the crystallization temperature of lipids is approximately 14° C., hence the therapeutic window of using cooling to selectively target tissues is equal or less than this temperature. Based on the data in Table 2, the estimated slurry injection could provide therapeutic effects for approximately 315 seconds.

TABLE 2
Estimated Skin Temperature Post Slurry Injection, Sample
slurry: −2.8° C., 50% Ice Content

| Time Post Cooling (sec) | Estimated Skin Temp (° C.) |
|---|---|
| 5 | 5.84 |
| 15 | 4.9 |
| 30 | 3.64 |
| 60 | 1.66 |
| 90 | 0.4 |
| 120 | −0.14 |
| 150 | 0.04 |
| 180 | 0.94 |
| 240 | 4.9 |
| 300 | 11.74 |
| 315 | 13.9* |
| 360 | 21.46 |
| 420 | 34.06 |

Ice Content Key Determinant of Cooling Capacity

Slurries of similar temperature and composition but different ice contents have drastically different cooling capacities. The graph of FIG. 28 depicts surface skin temperature post injection of normal saline with 10% glycerol slurry at −2.5° C. and approximately 10% ice content compared to −2.8° C. and 50% ice content. As shown in FIG. 28, the skin temperature reached a substantially lower temperature (i.e., ˜−3° C. vs. ˜12° C.) post-injection when injected with a 50% ice content slurry when compared to a 10% ice content slurry.

Experimental Data Used in Modeling

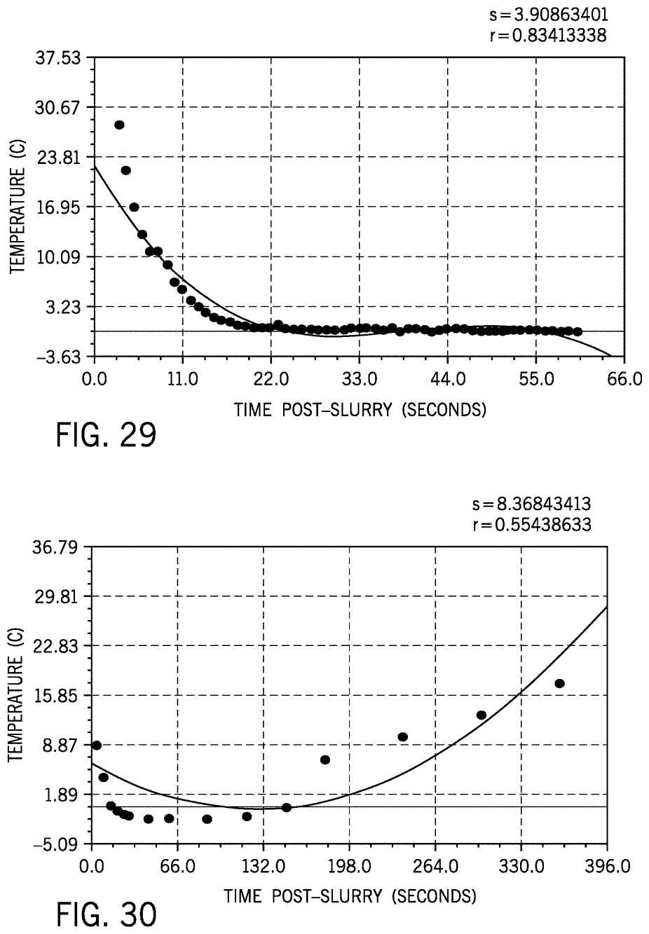

A best fit polynomial regression was implemented to model cooling characteristics of the first 60 seconds of slurry injection, as shown in FIG. 29. A best fit quadratic regression model was implemented to model cooling characteristics of slurry injection and subsequent rewarming, as shown in FIG. 30.

Fractional Cooling Experiments

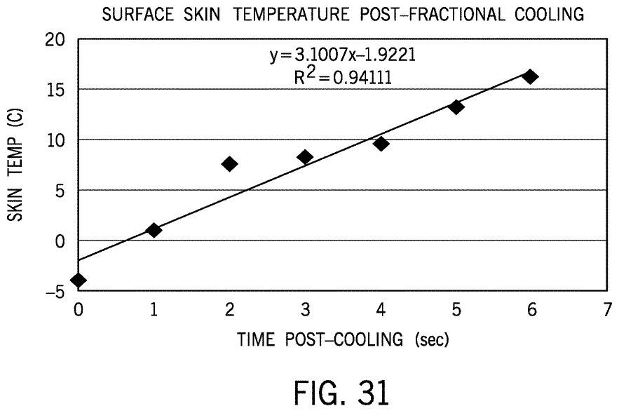

Following conductive cooling with the injection of needles in a fractional pattern at a 5 millimeter (mm) depth with 2 mm between needles (approximately 0.5 mm in diameter), rapid rewarming was observed. The needles were at a temperature of approximately −20° C. at time of insertion into skin. After approximately 1 minute of cooling, the cooling area of below 14° C. was approximately 0.301 cm². FIG. 31 illustrates the surface skin temperature as a function of time post-injection for the fractional needle test.

Based on the experiments conducted with slurry injections, it can be determined that 5 minutes of cooling below 14° C. may be sufficient to achieve selective destruction of lipid rich tissues. Hence, delivery of conductive cooling should be within this range. In order to maintain a fractionated pattern, multiple short cooling cycles can be implemented.

Table 3 below shows the bulk cooling parameters for the stimulation of blood vessels and neocollagensis. Of note, cooling capacity to target tissue at higher temperatures will be controlled primarily through adjusting injection volume, ice particle size, ice content, etc., as slurry temperature cannot be higher than 4° C.

TABLE 3
Bulk Cooling Parameters for stimulation of blood vessels and neocollagenesis

| | Temp of Tissue to | Temp of Tissue to be | |
|---|---|---|---|
| | be Targeted- | Targeted-Based on | |
| | Based on | Adipocyte Cooling | |
| | Selective Lipid | Below Physiologic | |
| Cooling Method | Targeting | Temperature | Cooling Durations |
| CryoSlurry-Bulk | −20 C. to +14 C.* | −20 C. to +30 C.* | Injected at Desired Site of |
| | | | Action: 1-10 minutes/ |
| | | | treatment cycle |
| | | | Topically Applied: 5-30 |
| | | | minutes/treatment cycle (to |
| | | | allow for diffusion of heat to |
| | | | deep dermis/superficial |
| | | | epidermis) |
| Other Conductive | | | Injected at Desired Site of |
| Cooling (TE, | | | Action (ie cooled |
| Spray)-Bulk | | | microneedles): 1-10 |
| | | | minutes/treatment cycle |
| | | | Topically Applied (ie non- |
| | | | penetrating cooling array): |
| | | | 5-30 minutes/treatment |
| | | | cycle (to allow for diffusion |
| | | | of heat to deep dermis/ |
| | | | superficial epidermis) |

Fractional Cooling Parameters for Stimulation of Blood Vessels and Neocollagenesis Using CryoSlurry

Table 4 illustrates experimental data to determine the maximum thermal radius of 10 mL CryoSlurry injections was performed, with a target 5 min treatment time using 50% ice content. Of note, slurry may spread differently in different tissue types and have different cooling capacities based on ice content, and this is only one non-limiting example. The tissue type tested was subcutaneous injection in a rat model. Also, injections may be placed closer than outlined parameters to achieve more uniform bulk cooling in a treatment area that injection volume via single injection.

TABLE 4
Experimental Data for Fractional Cooling Parameters for Stimulation
of Blood Vessels and Neocollagenesis using CryoSlurry

| | | | | | | Maximum |
|---|---|---|---|---|---|---|
| | | | | Minimum Inter- | | Number of Fractional |
| Slurry | | | | Injection Distance to | | Injection Sites to |
| Injection | | Thermal | | Maintain Fractional | | Prevent Fluid Overload |
| Volume | | Radius | | Pattern (Thermal | | (500 cc maximum) based |
| (cc) | | (cm) | | Diameter)-(cm) | | on Grid Pattern |
| 0.5 | cc | 0.05-0.1 | cm | 0.1-0.2 | cm | 1000 |
| 1.0 | cc | 0.1-0.2 | cm | 0.2-0.4 | cm | 500 |
| 2.0 | cc | 0.2-0.4 | cm | 0.4-0.8 | cm | 250 |
| 5.0 | cc | 0.5-1 | cm | 1.0-2.0 | cm | 100 |
| 10.0 | cc | 1-2 | cm | 2-4 | cm | 50 |
| 15.0 | cc | 1.5-3.0 | cm | 3-6 | cm | 33 |
| 20.0 | cc | 2-4 | cm | 4-8 | cm | 25 |

Cooling Times and Temperatures for Stimulation of Blood Vessels and Neocollagenesis Using Penetrating Needle Array or Topical Fractional Cooling Needle Arrays

As shown in Table 5, cycle time is longer for topical application, as it takes longer for cooling to diffuse to target site of deep dermis and superficial fat. Longer cycles are enabled by active rewarming to help maintain fractionated pattern and prevent bulk tissue effects. Given data described above showing rapid rewarming, there should be a minimum of 5 seconds between cycles.

TABLE 5
Cooling times and temperatures for stimulation of blood vessels formation and neocollagenesis
using penetrating needle array or topical fractional cooling needle arrays

| | | | | | | | Topical | | | |
|---|---|---|---|---|---|---|---|---|---|---|
| | | | | | | | Fractional | | | |
| | | | Micro needle | | Micro needle | | Cooling (non- | | Topical Fractional | |
| | | | cooling | | cooling | | penetrating)- | | Cooling (non- | |
| | | | (penetrating)- | | (penetrating) - | | No Active | | penetrating)- | |
| | | | No Active | | Active | | warming | | No Active warming | |
| | | | Warming at | | Warming at | | between cooling | | between cooling | |
| | | | Surface | | Surface | | sites | | sites | |
| Cycle Treatment Time | | | 10 sec-10 min | | 10 sec-20 min | | 10 sec-20 min | | 10 sec-10 min | |
| Number of | 1 | min | 1-6 | cycles | 1-6 | cycles | 1-6 | cycles | 1-6 | cycles |
| cycles to | 5 | min | 1-30 | cycles | 1-30 | cycles | 1-30 | cycles | 1-30 | cycles |
| achieve | 10 | min | 1-60 | cycles | 1-60 | cycles | 1-60 | cycles | 1-60 | cycles |
| cooling at | 20 | min | 2-120 | cycles | 2-120 | cycles | 1-120 | cycles | 2-120 | cycles |
| target site to | 30 | min | 3-180 | cycles | 2-180 | cycles | 2-180 | cycles | 3-180 | cycles |

achieve
cooling time
of . . .

Fractional Cooling Experiment on Mouse Skin

Ex-vivo mouse skin was tested to monitor the cooling temperature and efficiency of a cooling treatment system configured to achieve a fractional cooling pattern according to the present disclosure. A cooling treatment system was fabricated that included a delivery device having a plurality of copper needles extending from a plate. For the test, the delivery device included thirteen needles arranged in a 3-2-3-2-3 array pattern. The needles were spaced between 4 mm and 7 mm from one another and the needle diameter was between 1 mm and 1.3 mm. A Peltier cooler was thermally coupled to the plurality of copper needles to control an amount of cooling provided by the fabricated cooling treatment system. For the test, the Peltier cooler was configured to maintain the cooling treatment system at approximately −10° C.

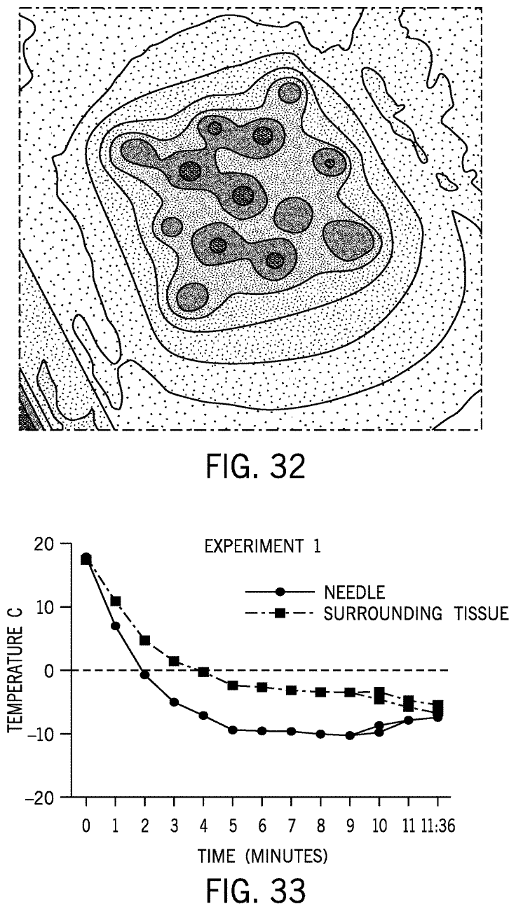

The mouse skin was placed on top of the copper needle array and the temperature was monitored from above using an forward looking infrared (FLIR) camera. As illustrated in FIG. 32, the cooling treatment system achieved a fractionated cooling pattern on the mouse skin with discrete zones of cooling surrounded by areas of higher temperature tissue (darker shading in FIG. 32 illustrates regions of lower temperature). Using the mouse skin temperature monitored by the FLIR camera, the temperature at the cooper needle sites and the surrounding tissue was calculated as a function of time. As illustrated in FIG. 33, the temperature of the tissue surrounding the needle mimicked the temperature profile of the needle as a function of time with the temperature continually approaching the needle temperature. This temperature response of the surrounding tissue demonstrates the feasibility and efficiency of using fractional cooling to cool the skin and underlying tissue.

Fractional Slurry Injection Experiment on Human Post Abdominoplasty Specimen

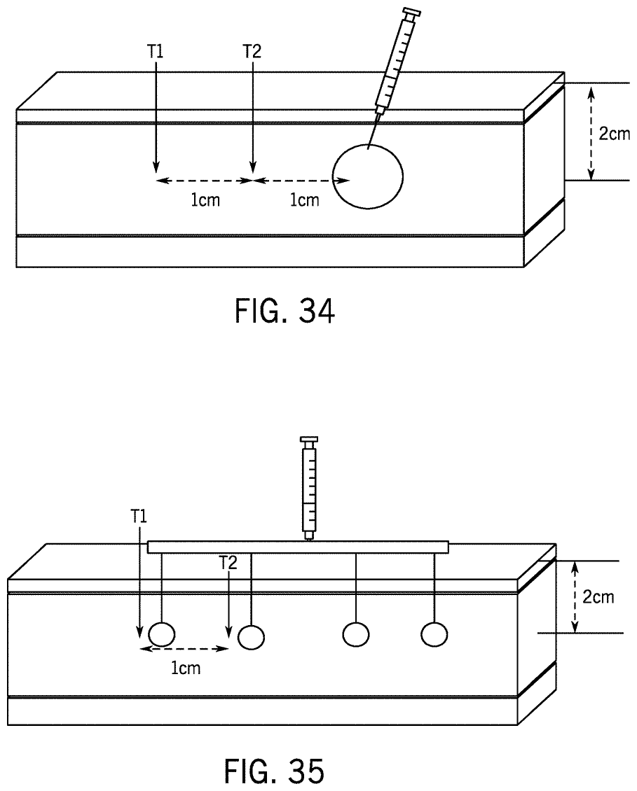

Human post abdominoplasty tissue was tested to compare cooling treatment of a single slurry injection and a fractional slurry injection. For the single slurry injection test, 60 mL of slurry was injected into subcutaneous fat of human post abdominoplasty tissue. The slurry temperature was approximately −4.8° C. and was composed of saline and 10% glycerol. As illustrated in FIG. 34, a first thermocouple (T1) was placed 1 centimeter (cm) laterally away from the injection site and 2 cm below a surface of the skin, and a second thermocouple (T2) was place 2 cm laterally away from the injection site (in the same direction as T1) and 2 cm below the surface of the skin. For the fractional slurry injection test, 60 mL of slurry was injected into subcutaneous fat of human abdominoplasty tissue using a device similar to the delivery device 102 of FIG. 16B. The slurry temperature was approximately −4.8° C. and was composed of saline and 10% glycerol. As illustrated in FIG. 35, a first thermocouple (T1) and a second thermocouple (T2) were placed 1 cm away from each other and adjacent and 2 cm below the surface of the skin.

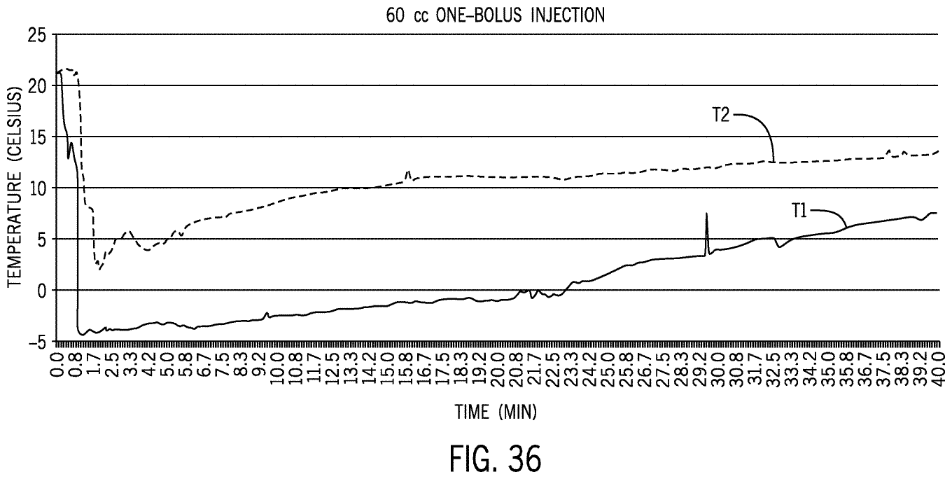

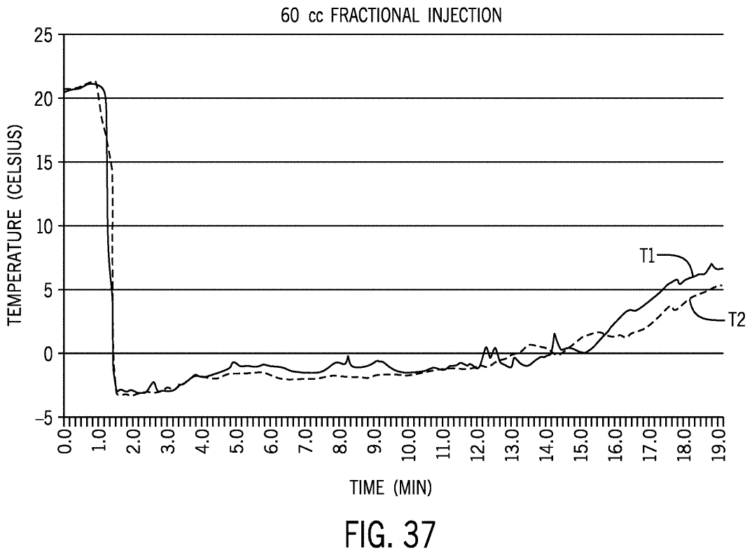

In both tests, the total volume of slurry was constantly injected into the subcutaneous fat and the temperature of T1 and T2 were monitored and recorded. As illustrated in FIG. 36, for the single slurry injection, the temperature measured by T2 was consistently warmer than the temperature measured by T1. This is due to the reduced cooling uniformity provided by a single bulk injection. Turning to FIG. 37, for the fractional slurry injection, both T1 and T2 measured approximately the same temperature for the duration of the injection process. This suggests that fractional slurry injections provide an increased cooling uniformity and covers a larger tissue area. In addition, a time required to complete the single bulk injection was approximately twice as long as the time required to inject the same volume of slurry with the fractional injection.

Thus, while the invention has been described above in connection with particular embodiments and examples, the invention is not necessarily so limited, and that numerous other embodiments, examples, uses, modifications and departures from the embodiments, examples and uses are intended to be encompassed by the claims attached hereto. The entire disclosure of each patent and publication cited herein is incorporated by reference, as if each such patent or publication were individually incorporated by reference herein.

We claim:

1. A cooling treatment system for applying cooling therapy to a desired tissue region of a patient, the cooling treatment system comprising:
a cooling device; and
a delivery device configured to be cooled by the cooling device and subject the desired tissue region to a desired temperature provided by the cooling device;
wherein the desired temperature is between approximately minus 200 degrees Celsius and approximately 30 degrees Celsius.

2. The cooling treatment system of claim 1, wherein the delivery device includes one or more protrusions configured to engage the desired tissue region

3. The cooling treatment system of claim 2, wherein the one or more protrusions comprise a needle conductively coupled to the cooling device.

4. The cooling treatment system of claim 2, wherein the one or more protrusions comprise a needle array conductively coupled to the cooling device.

5. The cooling treatment system of claim 2, wherein the one or more protrusions each comprise a needle including insulation wrapped axially around the needle, and wherein a needle tip of the needle is uninsulated.

6. The cooling treatment system of claim 2, wherein the one or more protrusions comprise a needle configured to inject a slurry.

7. The cooling treatment system of claim 2, wherein the one or more protrusions comprise a needle array configured to inject a slurry.

8. The cooling treatment system of claim 2, wherein the one or more protrusions each comprise a needle including an inlet passage and an outlet passage arranged therein, and wherein the inlet passage and outlet passage are configured to receive a flow of fluid to actively cool the needle.

9. The cooling treatment system of claim 1, wherein the one or more protrusions comprise an array configured to apply a slurry topically.

10. The cooling treatment system of claim 1, wherein the delivery device comprises an expandable needle including a balloon expandable between an inflated position and a deflated position.

11. The cooling treatment system of claim 1, further comprising a warming device.

12. The cooling treatment system of claim 11, wherein the warming device is coupled to a base of the delivery device adjacent to a proximal end of the one or more protrusions to heat a surface of the desired tissue region.

13. The cooling treatment system of claim 11, wherein the warming device comprises at least one of a radio frequency warming device and an infrared laser.

14. The cooling treatment system of claim 1, further comprising a depth imaging device configured to monitor a depth of the delivery device within the desired tissue region.

15. The cooling treatment system of claim 1, further comprising a thermal imaging device configured to monitor a temperature of a desired tissue region.

16. The cooling treatment system of claim 1, further comprising one or more temperature sensors configured to monitor a temperature of the delivery device and/or the desired tissue region.

17. The cooling treatment system of claim 1, wherein the desired temperature is between approximately minus 180 degrees Celsius and approximately 30 degrees Celsius.

18. The cooling treatment system of claim 1, wherein the desired temperature is between approximately minus 160 degrees Celsius and approximately 30 degrees Celsius.

19. The cooling treatment system of claim 1, wherein the desired temperature is between approximately minus 140 degrees Celsius and approximately 30 degrees Celsius.

20. The cooling treatment system of claim 1, wherein the desired temperature is between approximately minus 120 degrees Celsius and approximately 30 degrees Celsius.

21. The cooling treatment system of claim 26, wherein the desired temperature is between approximately minus 100 degrees Celsius and approximately 30 degrees Celsius.

22. The cooling treatment system of claim 1, wherein the desired temperature is between approximately minus 80 degrees Celsius and approximately 30 degrees Celsius.

23. The cooling treatment system of claim 1, wherein the desired temperature is between approximately minus 70 degrees Celsius and approximately 30 degrees Celsius.

24. The cooling treatment system of claim 1, wherein the desired temperature is between approximately minus 60 degrees Celsius and approximately 30 degrees Celsius.

25. The cooling treatment system of claim 1, wherein the desired temperature is between approximately minus 50 degrees Celsius and approximately 30 degrees Celsius.

26. The cooling treatment system of claim 1, wherein the desired temperature is between approximately minus 40 degrees Celsius and approximately 30 degrees Celsius.

27. The cooling treatment system of claim 1, wherein the desired temperature is between approximately minus 30 degrees Celsius and approximately 30 degrees Celsius.

28. The cooling treatment system of claim 1, wherein the desired temperature is between approximately minus 20 degrees Celsius and approximately 30 degrees Celsius.

29. The cooling treatment system of claim 1, wherein the desired temperature is between approximately minus 20 degrees Celsius and approximately 20 degrees Celsius.

30. The cooling treatment system of claim 1, wherein the desired temperature is between approximately minus 20 degrees Celsius and approximately 20 degrees Celsius.

31. The cooling treatment system of claim 1, wherein the desired temperature is between approximately minus 20 degrees Celsius and approximately 10 degrees Celsius.

32. The cooling treatment system of claim 1, wherein the desired temperature is between approximately minus 20 degrees Celsius and approximately 5 degrees Celsius.

33. The cooling treatment system of claim 1, wherein the delivery device further comprises a manifold configured to be removably coupled to one or more needles.

34. The cooling treatment system of claim 33, wherein the manifold includes an inlet port configured to be removably coupled to a slurry injection device.

35. The cooling treatment system of claim 34, wherein the manifold is configured to provide fluid communication between the inlet port and the one or more needles coupled thereto.

36. The cooling treatment system of claim 1, where the system is further configured to elicit an angiogenesis, a cryolipolysis, or a collagen remodeling response in the patient.

37. A method for causing angiogenesis in a subject, the method comprising:
identifying treatment parameters for a desired tissue region of the subject for receiving a treatment including cooling, using a cooling device, to a desired temperature provided by the cooling device, wherein the treatment parameters are based in part on at least one of the desired treatment tissue region or the treatment;
applying the treatment using the treatment parameters; and
eliciting an angiogenesis response of the desired treatment tissue to the treatment.

38. The method of claim 37, wherein the treatment includes a fractionated cooling process.

39. The method of claim 38, wherein the fractionated cooling process is a fractional slurry injection.

40. The method of claim 38, wherein the fractionated cooling process is a conductive cooling via a fractional needle array.

41. The method of claim 37, wherein the treatment includes an ablative cryotherapy.

42. The method of claim 37, wherein the treatment includes a bulk cooling process.

43. The method of claim 37, wherein the desired treatment tissue includes an ischemic organ or tissue.

44. The method of claim 43, wherein the ischemic organ or tissue includes one of burn scars, scars, keloids, aged skin, tissue influenced by diabetic neuropathy, a lipoma, cellulite, ischemically-damaged tissue, chronic wounds, tissue influenced by male pattern baldness, heart, liver, or kidney.

45. The method of claim 37, wherein the treatment is part of a vaginal rejuvenation treatment process, a skin rejuvenation treatment process, or an onychomycosis treatment process.

46. The method of claim 37, further comprising eliciting an collagen remodeling or a cryolipolysis response in the subject.

47. The method of claim 37, wherein the cooling device includes the system of claim 1.

48. A method for causing collagen remodeling in a subject, the method comprising:
identifying treatment parameters for a desired tissue region of the subject for receiving a treatment including cooling, using a cooling device, to a desired temperature provided by the cooling device, wherein the treatment parameters are based in part on at least one of the desired treatment tissue region or the treatment;
applying the treatment using the treatment parameters; and
eliciting a collagen remodeling response of the desired treatment tissue to the treatment.

49. The method of claim 48, wherein the treatment includes a fractionated cooling process.

50. The method of claim 48, wherein the treatment includes an ablative cryotherapy.

51. The method of claim 48, wherein the treatment includes a bulk cooling process.

52. The method of claim 48, wherein the desired treatment tissue includes tissue experiencing laxity.

53. The method of claim 52, wherein the desired treatment tissue includes one of burn scars, scars, keloids, aged skin, cellulite or chronic wounds.

54. The method of claim 48, wherein the treatment is part of a vaginal rejuvenation treatment process or a skin rejuvenation treatment process.

55. The method of claim 48, wherein the desired treatment tissue includes soft tissues of an airway of the subject.

56. The method of claim 55, wherein the treatment is part of an obstructive sleep apnea improvement process.

57. The method of claim 48, further comprising eliciting an angiogenesis or a cryolipolysis response in the subject.

58. The method of claim 48, wherein the cooling device includes the system of claim 1.

59. A method for causing cryolipolysis in a subject, the method comprising:
identifying treatment parameters for a desired tissue region of the subject for receiving a treatment including cooling, using a cooling device, to a desired temperature provided by the cooling device, wherein the treatment parameters are based in part on at least one of the desired treatment tissue region or the treatment, and the desired temperature is between approximately minus 200 degrees Celsius and approximately 30 degrees Celsius;
applying the treatment using the treatment parameters; and
eliciting a cryolipolysis response of the desired treatment tissue to the treatment.

60. The method of claim 59, wherein the treatment includes a fractionated cooling process.

61. The method of claim 59, wherein the treatment includes an ablative cryotherapy.

62. The method of claim 59, wherein the treatment includes a bulk cooling process.

63. The method of claim 59, wherein the desired treatment tissue includes one of a tongue or tissue in an airway of the subject.

64. The method of claim 59, wherein the treatment is part of obstructive sleep apnea improvement process.

65. The method of claim 59, further comprising eliciting an angiogenesis or a collagen remodeling response in the subject.

66. The method of claim 59, wherein the cooling device includes the system of claim 1.
